(19) United States
(12) Patent Application Publication
(10) Pub. No.: US 2014/0316520 A1
(43) Pub. Date: Oct. 23, 2014

(54) SUPPORT SEGMENT FOR OCULAR STRUCTURES

(71) Applicant: Accutome, Inc., Malvern, PA (US)

(72) Inventors: Allon Barsam, London (GB); Eric D. Donnenfeld, Roslyn, NY (US)

(73) Assignee: Accutome, Inc., Malvern, PA (US)

(21) Appl. No.: 14/257,957

(22) Filed: Apr. 21, 2014

Related U.S. Application Data
(60) Provisional application No. 61/813,862, filed on Apr. 19, 2013.

(51) Int. Cl. A61F 2/14

(52) U.S. Cl. CPC A61F 2/14
USPC 623/4.1

(57) ABSTRACT

Devices and methods for providing capsular support to areas of zonular weakness and for iris dilation in complex cataract surgery and other ophthalmological procedures. In one embodiment, a device for implantation into an eye includes at least one engagement feature and at least one attachment mechanism. The engagement feature is adapted to engage with the edge of an ocular structure, e.g., the anterior edge of the capsular bag or an iris. The attachment mechanism, e.g., a hole, is coupled to the engagement feature and adapted to receive an anchor, e.g., an iris hook or suture, that biases the device towards the edge of the ocular structure.

### CROSS-REFERENCE TO RELATED APPLICATION

This application claims priority to co-pending U.S. Provisional Patent Application Ser. No. 61/813,862, filed Apr. 19, 2013, the disclosure of which is incorporated herein by reference in its entirety.

### BACKGROUND

The disclosure relates, generally, to eye surgery, and more particularly, to devices for providing support to structures of the eye in connection with cataract surgery and other ophthalmologic procedures.

In complex cataract surgery and other ophthalmologic procedures, the natural lens-supporting system of patients is often compromised due to generalized weakness of, or extensive local defects of zonules, which are fibrous strands in a ring formation that connect circumferential tissue inside the eye known as the ciliary body with the crystalline lens of the eye. Zonular laxity, zonular dehiscence, and other zonular defects and deficiencies can be caused by a number of factors and conditions, including, e.g., trauma, pseudoexfoliation syndrome, glaucoma, high myopia, and hereditary systemic diseases. Without proper stabilization, such zonular weakness can increase the risk of surgical complications, including capsular tears, vitreous prolapse, and intraocular-lens (IOL) instability.

A number of devices for improving zonular weakness have been developed.

For example, capsular-tension rings, such as Cionni rings and Morcher rings, provide good support to the capsular equator but provide insufficient anterior capsular support. If capsular-tension rings are inserted prior to irrigation and aspiration, they can impede the removal of soft lens matter and lead to further zonular stress due to the increased force needed to remove them.

Capsular-tension segments, such as Ahmed segments, provide good anterior capsular support but provide insufficient equatorial support.

Capsular-tension hooks, which are iris hooks modified to retract the capsular edge, do not always provide sufficient fixation and often become dislodged during phacoemulsification (i.e., cataract surgery in which the eye's internal lens is emulsified with an ultrasonic handpiece and aspirated from the eye). Capsular hooks can also give rise to anterior capsular tears, which, if allowed to propagate, can run beyond the capsular equator and cause dropped nuclear material, vitreous loss, and the inability to place an intraocular-lens (IOL) in the capsular bag or even in the sulcus.

Additionally, such capsular-tension rings, segments, and hooks can all be difficult to manipulate during surgery. Moreover, such devices are intended for temporary implantation during surgery and require removal prior to the conclusion of surgery.

### SUMMARY

Embodiments of the disclosure provide solutions to the foregoing problems and additional benefits, by providing devices and methods for providing capsular support in areas of zonular weakness or dialysis.

In one embodiment, the present disclosure provides a device for implantation into an eye. The device includes at least one engagement feature and at least one attachment mechanism. The engagement feature is adapted to engage with the edge of an ocular structure. The attachment mechanism is coupled to the engagement feature and adapted to receive an anchor that biases the device towards the edge of the ocular structure.

In another embodiment, the present disclosure provides a method for supporting an ocular structure of the eye. The method includes: inserting, into the eye, a device comprising at least one engagement feature adapted to engage with the edge of an ocular structure, and at least one attachment mechanism coupled to the engagement feature and adapted to receive an anchor; engaging the engagement feature with the edge of the ocular structure; and biasing the device towards the edge of the ocular structure by attaching the anchor to the device via the at least one attachment mechanism.

In a further embodiment, the present disclosure provides a device for implantation into an eye. The device includes a generally arcuate segment having a front portion and a rear portion. The front portion has an inner curvature and at least one aperture formed near the inner curvature. The rear portion has an outer curvature and at least one rear-facing depression formed near the outer curvature and has a surface with a generally U-shaped, V-shaped, or C-shaped cross section.

### BRIEF DESCRIPTION OF THE DRAWINGS

FIG. 1 shows a plan view of a capsular-tension segment consistent with a first exemplary embodiment of the disclosure;

FIG. 2 shows a front elevational view of the capsular-tension segment of FIG. 1;

FIG. 3 shows a side perspective view of the capsular-tension segment of FIG. 1;

FIG. 4 shows a step of inserting the capsular-tension segment of FIG. 1 into the anterior chamber of the eye through an incision in the capsular bag;

FIG. 5 shows a step of positioning the capsular-tension segment of FIG. 1 within the anterior chamber of the eye towards an area of zonular laxity;

FIG. 6 shows a step of securing the capsular-tension segment of FIG. 1 in place using an iris hook;

FIG. 7 shows a step of securing the capsular-tension segment of FIG. 1 in place using three iris hooks;

FIG. 8 shows a step of securing a second capsular-tension segment of FIG. 1 in place within the anterior chamber of the eye using an iris hook;

FIG. 9 shows a step of securing the capsular-tension segment of FIG. 1 in place using polypropylene suture;

FIG. 10 shows a plan view of a capsular-tension segment consistent with a second exemplary embodiment of the disclosure;

FIG. 11 shows a front elevational view of the capsular-tension segment of FIG. 10;

FIG. 12 shows a plan view of a capsular-tension segment consistent with a third exemplary embodiment of the disclosure; and

FIG. 13 shows a step of inserting an iris hook into the capsular-tension segment of FIG. 12.

### DESCRIPTION

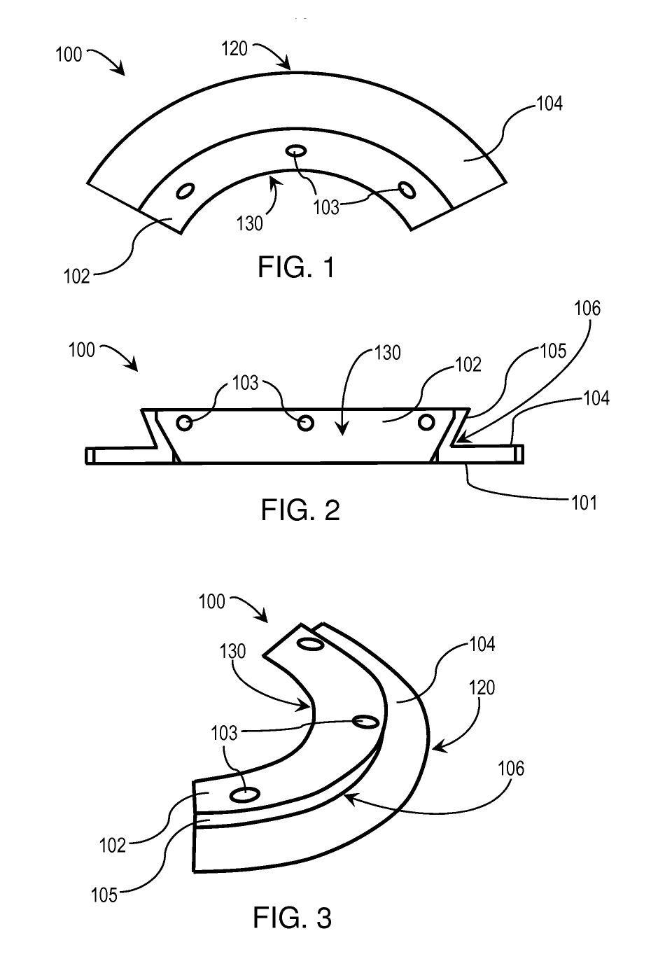

With reference first to FIGS. 1-3, a capsular-tension segment 100 consistent with a first exemplary embodiment of the disclosure is illustrated. As shown, segment 100 is generally arc-shaped and includes a bottom surface 101, a top surface 102 with three attachment holes 103 formed therein, a lower engagement surface 104, an upper engagement surface 105, and a groove 106.

Segment 100 is formed from milled medical-grade polymethyl methacrylate (PMMA) or another inert and biocompatible material suitable for fabrication of intraocular devices.

Segment 100 has a generally V-shaped cross section, with a wider lower engagement surface 104 that protrudes from the rear 120 of segment 100 and a narrower top surface 102 that tapers downward toward the front 130 of segment 100. Lower engagement surface 104 has an outer curvature 114, and the front 130 of segment 100 has an inner curvature 134.

Bottom surface 101, top surface 102, and lower engagement surface 104 of segment 100 are all generally flat to permit segment 100 to slide around easily inside the capsular bag for positioning without disrupting the integrity of any portion of the capsular bag.

Lower engagement surface 104 is adapted to slide under the anterior capsular edge and extend toward the equatorial region, and upper engagement surface 105 is adapted to extend over the anterior capsular edge when lower engagement surface 104 is in place under the anterior capsular edge. Lower engagement surface 104 and upper engagement surface 105 are angled to form groove 106, which engages the anterior capsular edge (e.g., at an area of zonular laxity) when lower engagement surface 104 is in place under the anterior capsular edge, causing top surface 102 to protrude anterior to the anterior capsular edge and external to the capsular bag.

Attachment holes 103 are formed to be sufficiently large for an iris hook, suture, or other fixation device to pass through. One or more such fixation devices are passed through one or more of attachment holes 103 to secure segment 100 in place when lower engagement surface 104 is in place under the anterior capsular edge.

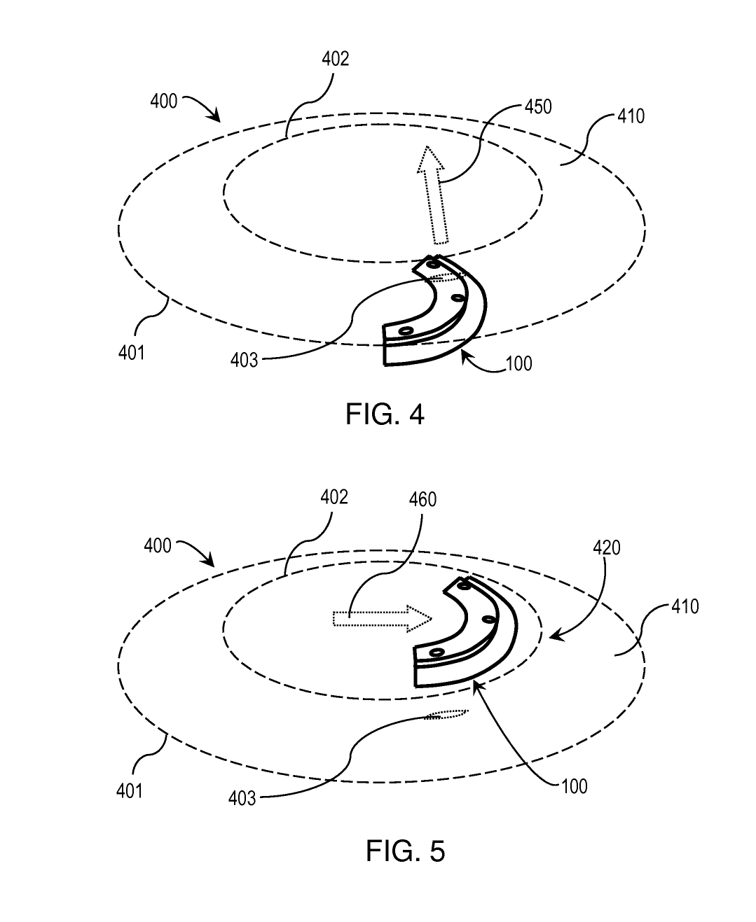

Turning now to FIGS. 4-6, a first exemplary method for using capsular-tension segment 100 in the anterior chamber 400 of a patient's eye will now be described. Broken lines are used to show the outline of the anterior portion of capsular bag 410, which is a sack-like structure remaining within the eye following certain ophthalmological procedures, such as extracapsular cataract extraction or phacoemulsification. Capsular bag 410 has an equatorial region 401, an annular anterior capsular edge 402 (also referred to as the “capsular rim,” “capsular flap,” or “capsular leaflet”), and a paracentesis incision 403 (or phacoemulsion wound, or other opening) created during the ophthalmological procedure.

First, as shown in FIG. 4, segment 100 is inserted into capsular bag 410 through incision 403, e.g., using standard or angled forceps, in the direction indicated by arrow 450. In an alternative embodiment, an injector may be used to insert segment 100.

Second, as shown in FIG. 5, segment 100 is slid around inside capsular bag 410, e.g., using standard or angled forceps, and maneuvered towards annular anterior capsular edge 402 in the direction indicated by arrow 460, i.e., towards an area 420 of zonular laxity. In an alternative embodiment, an injector may be used to position segment 100 within capsular bag 410.

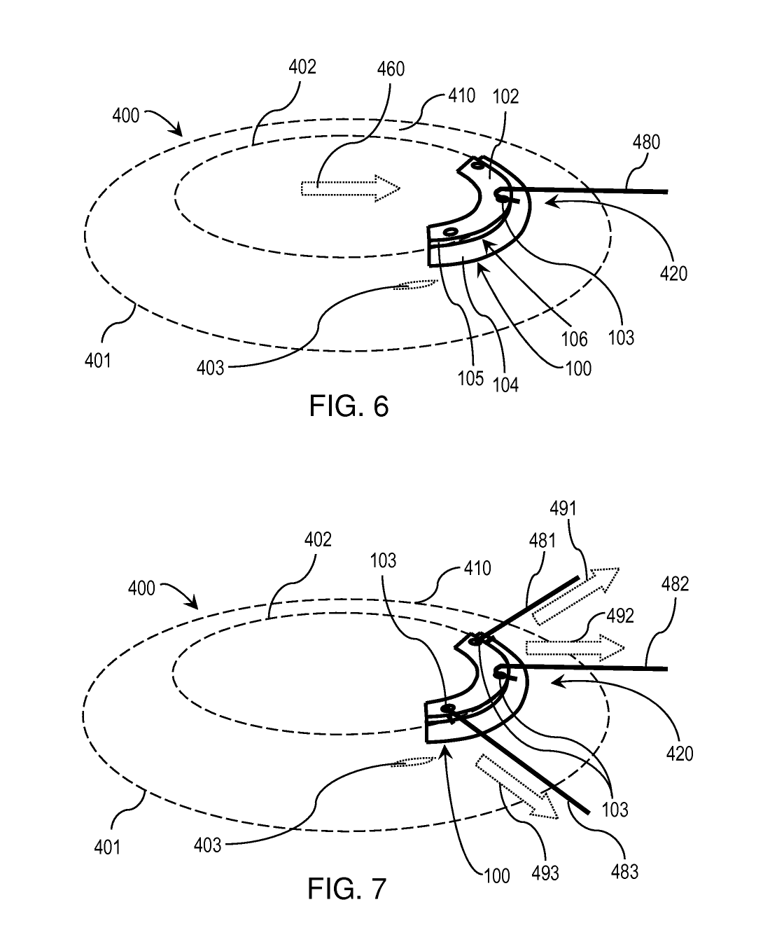

Third, as shown in FIG. 6, segment 100 is positioned to engage with annular anterior capsular edge 402 in the direction indicated by arrow 460, so that segment 100 can provide support to an area 420 of zonular laxity. More specifically, lower engagement surface 104 is slid under anterior capsular edge 402 so as to extend toward equatorial region 401, with upper engagement surface 105 extending over anterior capsular edge 402, such that groove 106 engages anterior capsular edge 402 at area 420 of zonular laxity, and top surface 102 protrudes anterior to anterior capsular edge 402 and external to capsular bag 410.

Fourth, an iris hook 480 is inserted into anterior chamber 400 in the direction opposite from that indicated by arrow 460, through an incision (not shown) made in the area of the transition area (i.e., corneal limbus) from the cornea to the sclera of the patient's eye.

Fifth, iris hook 480 is maneuvered to pass through the center attachment hole 103, and segment 100 is secured in place by applying tension to iris hook 480 in the direction indicated by arrow 460. Tension is applied, e.g., by clamping, onto iris hook 480, one or more fixation elements (not shown) that are positioned to hold iris hook 480 in place at the transition area. Such fixation elements may be integral to iris hook 480 or may be separate components.

FIG. 7 illustrates a second exemplary method for using capsular-tension segment 100 in the anterior chamber 400 of a patient's eye. As shown, the method of FIG. 7 is similar to the method of FIGS. 4-6, except that segment 100 is held in place with three iris hooks 481, 482, 483, where one of iris hooks 481, 482, 483 is respectively disposed in each of the three attachment holes 103 of segment 100. Just as in FIG. 6, iris hooks 481, 482, 483 are inserted into anterior chamber 400 through the same or different incisions (not shown) made in the transition area from the cornea to the sclera of the patient's eye. Each of iris hooks 481, 482, 483 is maneuvered to pass through a respective attachment hole 103, and segment 100 is secured in place by applying tension to all three iris hooks 481, 482, 483 in the respective directions indicated by arrows 491, 492, 493, e.g., by clamping, onto iris hooks 481, 482, 483, one or more fixation elements (not shown) that are positioned to hold iris hooks 481, 482, 483 in place at the transition area. It should be recognized that only two of the three attachment holes 103 may be used in alternative embodiments of the disclosure, and that other embodiments of a capsular-tension segment are possible having numbers of attachment holes other than three. Additionally, in alternative embodiments of a capsular-tension segment, alternative mechanisms for securing the capsular-tension segment may be used instead of attachment holes, e.g., hooks disposed on or integrated into the capsular-tension segment, polypropylene or other filament or wire disposed on or integrated into the capsular-tension segment, or the like.

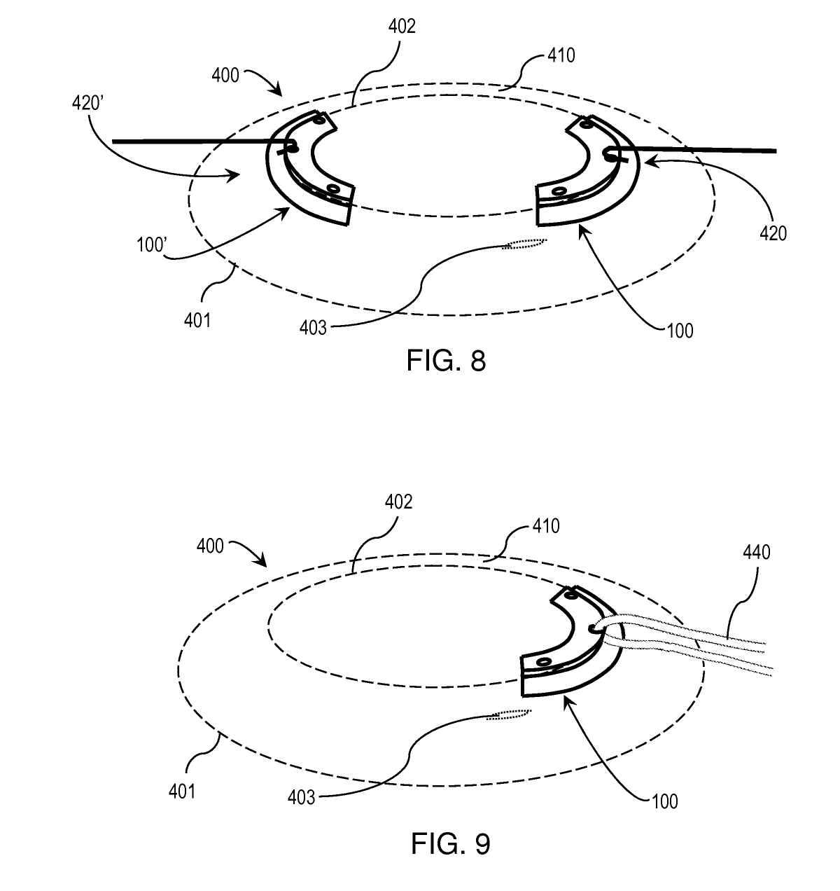

FIG. 8 illustrates a third exemplary method for using capsular-tension segment 100 in the anterior chamber 400 of a patient's eye. As shown, the method of FIG. 8 is similar to the method of FIGS. 4-6, except that two opposing capsular-tension segments 100, 100′ are installed into capsular bag 410, e.g., at two different areas 420, 420′ of zonular laxity. It should be recognized that more than one or two capsular-tension segments may be used in alternative embodiments of the disclosure, and that such capsular-tension segments may be used in various arrangements about annular anterior capsular edge 402. Two or more capsular-tension segments may be implanted to evenly distribute the force on the zonules, e.g., in eyes with extensive zonular laxity.

FIG. 9 illustrates a third exemplary method for using capsular-tension segment 100 in the anterior chamber 400 of a patient's eye. As shown, the method of FIG. 9 is similar to the method of FIGS. 4-6, except that capsular-tension segment 100 is secured to the sclera of the patient using a polypropylene suture line 440 (e.g., 9-0 or 10-0 Proline suture) through one of attachment holes 103, instead of using an iris hook. This configuration may be used during and even after surgery, whereby capsular-tension segment 100 can remain implanted in the eye of the patient postoperatively. However, it is desirable that a method as shown in FIGS. 4-6 be employed initially to secure capsular-tension segment 100 using an iris hook 480 during surgery, with the iris hook 480 then removed, and capsular-tension segment 100 then sutured into place, e.g., to improve postoperative stability and minimize the risk of anterior capsular phimosis following cataract surgery. It should be recognized that two or more of the attachment holes 103 may be used for attaching suture lines in alternative embodiments of the disclosure, and that other embodiments of a capsular-tension segment are possible having numbers of attachment holes for suture line other than three.

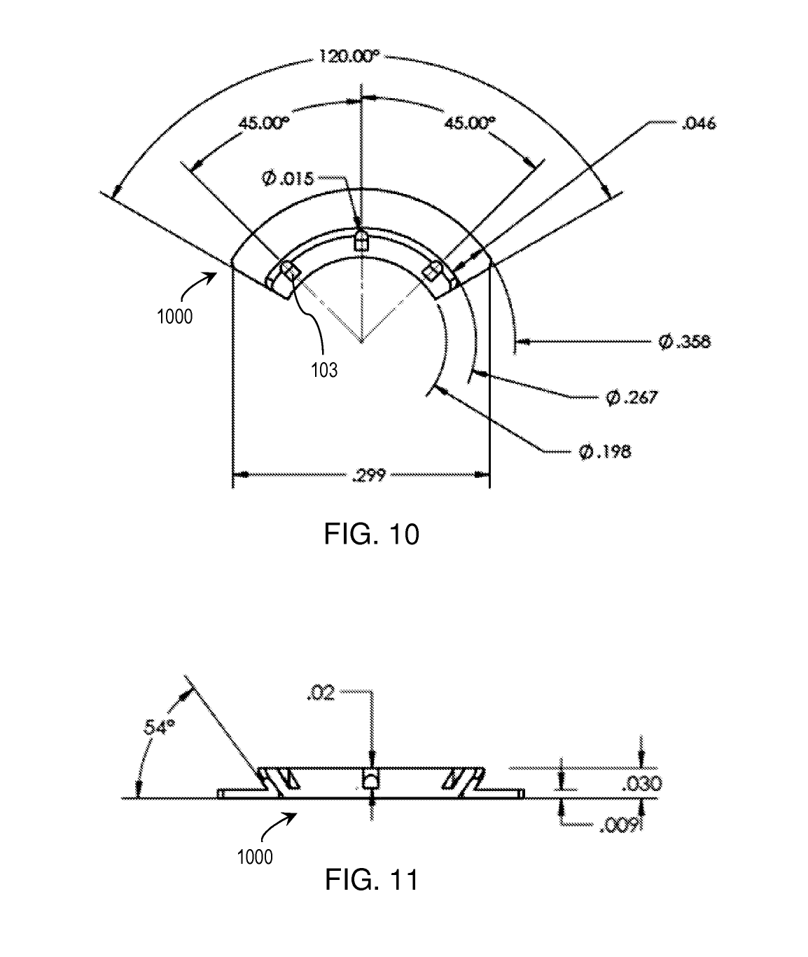

FIGS. 10 and 11 illustrate a capsular-tension segment 1000 consistent with a second exemplary embodiment of the disclosure. Segment 1000 is similar to segment 100, except that the attachment holes 1003 of segment 1000 are rectangular with one rounded end. FIGS. 10 and 11 illustrate exemplary dimensions (provided in inches) and angles (provided in degrees) for a capsular-tension segment consistent with certain embodiments of the disclosure, although such dimensions and angles should not be construed as limiting.

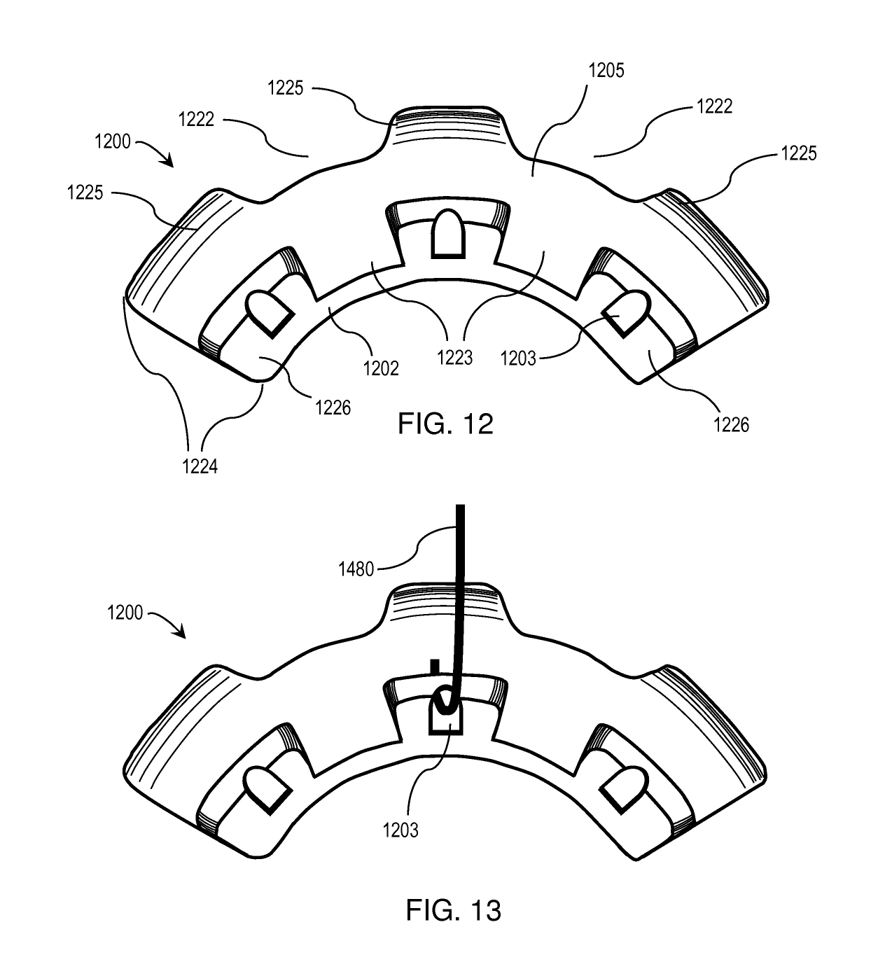

FIGS. 12 and 13 illustrate a capsular-tension segment 1200 consistent with a third exemplary embodiment of the disclosure. Segment 1200 is similar to segment 100, except that portions of the lower engagement surface (not visible in FIGS. 12 and 13) and upper engagement surface 1205 are notched at areas 1222, and portions of top surface 1202 are notched at areas 1223, so that less material is used during the fabrication of segment 1200. Additionally, the corners 1224 of segment 1200 are rounded, and attachment holes 1203 are rectangular with one rounded end. In the embodiment of FIGS. 12 and 13, due to notched areas 1222, the lower engagement surface (not visible in FIGS. 12 and 13) and upper engagement surface 1205 form three distinct “cuffs” 1225 that are spaced 45° apart and angled 54° (e.g., as shown in FIG. 11) relative to the bottom surface (not visible in FIGS. 12 and 13) of segment 1200. These three cuffs 1225 provide 120° of support to the anterior capsular edge distributed by the three cuffs 1224, while reducing the surface area that contacts the anterior capsular edge relative to the embodiments of FIGS. 1-11 Likewise, due to notched areas 1223, the top surface 1202 forms three distinct raised regions 1226 that are spaced 45° apart. As shown in FIG. 13, segment 1200 is secured in place using an iris hook 1480 through one of the attachment holes 1203.

Thus, embodiments of a capsular-tension segment have been described wherein the segment is adapted to engage the anterior capsular edge in order to provide support in areas of zonular weakness. The segment can be inserted into the capsular bag via an incision, maneuvered to the relevant area of zonular weakness, and secured via one or more standard iris hooks. The larger size of a capsular-tension segment consistent with embodiments of the disclosure, relative to an iris hook used alone, permits increased capsular stability and reduces the chance of unexpected dislodgement during surgical procedures. A capsular-tension segment consistent with embodiments of the disclosure can also be implanted permanently into the capsular bag using sutures, to provide permanent stability and support after surgery.

Although a capsular-tension segment consistent with embodiments of the disclosure is similar to a capsular-tension ring in that both provide support to the equator of the capsular bag, a capsular-tension segment consistent with embodiments of the disclosure provides the additional benefit of supporting the anterior capsule, which capsular-tension rings do not provide. Additionally, insertion of a capsular-tension ring is performed blindly, which may lead to malposition of the capsular-tension ring and capsular tears, while a capsular-tension segment consistent with embodiments of the disclosure is continuously visible during the entire procedure and can be inserted in a controlled manner, as described herein.

It should also be recognized that numerous other embodiments of applications and methods for using a capsular-tension segment consistent with embodiments of the disclosure exist, in addition to those described above. For example, a capsular-tension segment consistent with embodiments of the disclosure may have particular utility, not only in cataract surgery, but also in refractive lens exchange surgery, intraocular lens (IOL) repositioning surgery, and so forth.

In another exemplary application, a capsular-tension segment consistent with embodiments of the disclosure may be used as an iris retractor, to temporarily dilate the iris during surgery or other procedures. In this scenario, the capsular-tension segment is applied by sliding the lower engagement surface under the iris, with the upper engagement surface above the iris, and then fixating the segment with an iris hook via an incision (e.g., in the manner described above) in order to retract the iris in an area where the pupil does not dilate well. After using a segment to dilate the iris, the same segments can then be moved to the capsular bag and used to stabilize the anterior capsular edge, as described above.

A capsular-tension segment consistent with embodiments of the disclosure can also be used on an ad-hoc basis, e.g., to stabilize the anterior capsular edge in the event complications arise during cataract surgery. Because a capsular-tension segment consistent with embodiments of the disclosure can be inserted through an existing incision and involves no specialized instruments or techniques, even less experienced surgeons who may be faced with capsular complications can use the capsular-tension segment with ease.

Although embodiments of the disclosure describe a capsular-tension segment as being made from milled medical-grade polymethyl methacrylate (PMMA), it should be understood that other biocompatible materials may be used.

Although a number of features of embodiments of the disclosure are shown in the figures herein as having sharp corners, it should be understood that such sharp corners could alternatively be rounded, in alternative embodiments of the disclosure.

The term “ocular structure,” as used herein, should be understood to include the anterior capsular edge and the iris, as well as other structures of the eye that have edges and/or for which embodiments of the disclosure are suitable for stabilizing, supporting, and/or retracting.

The term “engagement feature,” as used herein, should be understood to include a groove (e.g., groove 106) with a V-shaped cross section formed at the interface between the lower engagement surface and the upper engagement surface, as well as any other mechanism that may be alternatively used for engagement with the anterior capsular edge, the iris, or another ocular structure, e.g., a flange, a ridge, a lip, a rim, an edge, a valley, a notch, a collar, a trough, another depression having a generally U-shaped, C-shaped, or V-shaped cross section, and other types of features. It should also be understood that, although embodiments are described wherein the engagement feature includes a lower engagement surface that slides under the anterior capsular edge and an upper engagement surface extends above the anterior capsular edge, in alternative embodiments, it is possible that the engagement feature includes only a lower engagement surface, or that the engagement feature includes only an upper engagement surface, wherein only that single surface is used to engage the anterior capsular edge. In some alternative embodiments, an engagement feature having a U-shaped, C-shaped, or V-shaped cross section, or other cross section, may be used to engage the anterior capsular edge laterally, without any portion of that feature actually extending under or above the anterior capsular edge.

The term “attachment mechanism” should be understood to include attachment holes, as described above, as well as other mechanisms for coupling a capsular-tension segment consistent with embodiments of the disclosure to an iris hook, a suture line, or other device used to apply tension to the capsular-tension segment to support, stabilize, and/or retract an ocular structure. Such mechanisms may include, e.g., hooks, loops, catches, pegs, links, clasps, hasps, and the like. The term “hole” should not be construed as requiring an aperture of a particular size, shape, or configuration, and includes fully-formed holes as well as slots and partially-open holes, such as a hole having a non-annular (e.g., C-shaped or partial helical) cross-section.

The term “filament” should be understood to include suture lines, wires, and other flexible, inflexible, or semiflexible devices capable of attachment to a capsular-tension segment consistent with embodiments of the disclosure via one or more attachment mechanism disposed thereon, which are used to secure the capsular-tension segment in place relative to the edge of an ocular structure (e.g., by attaching one end of the filament to the sclera of a patient's eye).

The term “anchor” should be understood to include iris hooks, filaments, and other flexible, inflexible, or semiflexible devices capable of attachment to a capsular-tension segment consistent with embodiments of the disclosure via one or more attachment mechanism disposed thereon, which are used to secure the capsular-tension segment in place relative to the edge of an ocular structure (e.g., by attaching one end of the anchor to the corneal limbus of the patient's eye).

Although embodiments of the disclosure are described as being used in connection with human eyes, it should be understood that a capsular-tension segment consistent with embodiments of the disclosure may alternatively be used with non-human animals, for veterinary applications.

It should be understood that various changes in the details, materials, and arrangements of the parts which have been described and illustrated in order to explain the nature of this disclosure may be made by those skilled in the art without departing from the scope of the disclosure. For example, it should be understood that the inventive concepts of embodiments of the invention may be applied to structures of the eye other than those specifically described herein.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments.

Although the disclosure has been described using relative terms such as “front,” “back,” “top,” “bottom,” “over,” “above,” “under” and the like in the description and in the claims, such terms are used for descriptive purposes and not necessarily for describing permanent relative positions. It is understood that the terms so used are interchangeable under appropriate circumstances such that the embodiments of the disclosure described herein are, for example, capable of operation in other orientations than those illustrated or otherwise described herein.

Although the disclosure is described herein with reference to specific embodiments, various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure. Any benefits, advantages, or solutions to problems that are described herein with regard to specific embodiments are not intended to be construed as a critical, required, or essential feature or element of any or all the claims.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the disclosure.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Although the disclosure has been set forth in terms of the exemplary embodiments described herein and illustrated in the attached documents, it is to be understood that such disclosure is purely illustrative and is not to be interpreted as limiting. Consequently, various alterations, modifications, and/or alternative embodiments and applications may be suggested to those skilled in the art after having read this disclosure. Accordingly, it is intended that the disclosure be interpreted as encompassing all alterations, modifications, or alternative embodiments and applications as fall within the true spirit and scope of this disclosure.

It will be further understood that various changes in the details, materials, and arrangements of the parts which have been described and illustrated in order to explain the nature of this disclosure may be made by those skilled in the art without departing from the scope of the disclosure as expressed in the following claims.

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

What is claimed is:

1. A device for implantation into an eye, the device comprising:
at least one engagement feature adapted to engage with the edge of an ocular structure; and
at least one attachment mechanism coupled to the engagement feature and adapted to receive an anchor that biases the device towards the edge of the ocular structure.

2. The device of claim 1, wherein the engagement feature includes a first surface disposed below the edge of the ocular structure when the engagement feature is engaged with the edge of the ocular structure.

3. The device of claim 2, wherein the engagement feature further includes a second surface disposed above the edge of the ocular structure when the engagement feature is engaged with the edge of the ocular structure.

4. The device of claim 1, wherein the ocular structure is a capsular bag, and the edge of the ocular structure is an anterior capsular edge.

5. The device of claim 1, wherein the ocular structure is an iris, and the edge of the ocular structure is an edge of the iris.

6. The device of claim 1, wherein the at least one engagement feature comprises two or more separate engagement features spaced apart from one another and adapted to engage respectively with two different portions of the edge of the ocular structure.

7. The device of claim 1, wherein the at least one engagement feature comprises a single contiguous engagement feature.

8. The device of claim 1, wherein the at least one engagement feature comprises an engagement feature having a generally arcuate curvature.

9. The device of claim 1, wherein the device is generally arcuate.

10. The device of claim 1, wherein the at least one attachment mechanism is a hole.

11. The device of claim 10, wherein the anchor is an iris hook.

12. The device of claim 10, wherein the anchor is a filament.

13. The device of claim 1, wherein the at least one attachment mechanism includes two or more attachment mechanisms coupled to the engagement feature, each attachment mechanism adapted to receive a respective anchor that biases the device towards the edge of the ocular structure.

14. The device of claim 1, wherein at least two of the anchors bias the device towards the edge of the ocular structure by applying tension to the device in two respective different directions.

15. A method for supporting an ocular structure of the eye, the method comprising:
inserting, into the eye, a device comprising at least one engagement feature adapted to engage with the edge of an ocular structure, and at least one attachment mechanism coupled to the engagement feature and adapted to receive an anchor;
engaging the engagement feature with the edge of the ocular structure; and
biasing the device towards the edge of the ocular structure by attaching the anchor to the device via the at least one attachment mechanism.

16. The method of claim 15, wherein the ocular structure is a capsular bag, and the edge of the ocular structure is an anterior capsular edge.

17. The method of claim 15, wherein the ocular structure is an iris, and the edge of the ocular structure is an edge of the iris.

18. The method of claim 15, wherein the anchor is an iris hook.

19. The method of claim 15, wherein the anchor is a filament.

20. A device for implantation into an eye, the device comprising:
a generally arcuate segment comprising:
a front portion having an inner curvature and at least one aperture formed near the inner curvature; and
a rear portion having an outer curvature and at least one rear-facing depression formed near the outer curvature and having a surface with a generally U-shaped, V-shaped, or C-shaped cross section.
